(19) United States
(12) Patent Application Publication
(10) Pub. No.: US 2002/0182162 A1
(43) Pub. Date: Dec. 5, 2002

(54) Nitric oxide (NO) donor+cGMP-PDE5 inhibitor as a topical drug for enhanced hair growth

(76) Inventors: Mohsen Shahinpoor, Albuquerque, NM (US); David Soltanpour, New York, NY (US); Parsa Shahinpoor, Albuquerque, NM (US)

Correspondence Address: MOHSEN SHAHINPOOR, 909 VIRGINIA, NE, SUITE 205, ALBERQUERQUE, NM, 87108, US

(21) Appl. No.: 10/064,698

(22) Filed: Aug. 7, 2002

(51) Int. Cl. A61K 9/20; A61K 7/06; A61K 7/11; A61K 31/715; A61K 31/496; A61K 38/00

(52) U.S. Cl. 424/70.13; 424/70.14; 514/2; 514/255.01; 514/54

(57) ABSTRACT

A new topical drug for enhancing hair growth or diminishing hair loss or alopecia, which comprises a mixture of a nitric oxide (NO) donor such as nitrovasodilators like minoxidil (Rogaine^{RTM}), nitroglycerin, L-arginine, isosorbide dinitrate, or nitroprusside, and a cyclic guanosine 3′,5′-monophosphate (cGMP) specific phosphodiesterase type 5 (PDE5) inhibitor such as sildenafil citrate (Viagra^{RTM}) in a dermatologically acceptable solution mix. In this manner there will be increased vascular circulation and blood circulation to the hair bulbs and follicles and the effect of the combined compounds is enhanced synergistically.

### BACKGROUND OF INVENTION

The Present invention provides a topical method of enhancing hair growth or diminishing hair loss or alopecia. Particularly, the invention provides a method, which can synergistically enhance vascular circulation and blood flow to hair follicles and bulbs.

The rate of hair growth and the length of its growth cycle are reduced with age. Those phenomena are common to all mammals with rare exceptions, and they must be differentiated from true male pattern alopecia, which is caused by target organ sensitivity to androgens. Compared to most epithelial structures, the hair follicle does not grow continuously throughout its life, but passes through a cycle called the pilar cycle. The pilar cycle is essentially comprised of three phases called the anagen or growth phase during which hair is produced, normally lasting about three to seven years; the catagen phase when growth stops and the follicle atrophies, lasting about three to four weeks; and the telogen phase, which is a rest period for the follicle during which the hair progressively separates and finally falls out, and normally lasting about three to four months. The anagen phase comprises nearly 95 percent of the follicle phase, less than 1 percent being in the catagen phase, and the rest being in the telogen phase. When the pilar cycle is disturbed, alopecia results followed by excessive hair loss. This anagen phase of the pilar cycle has different origins, such as febrile conditions, mental stresses, hormonal problems and secondary effects of drugs. Alopecia may also be due to age and to a slowing down of mitotic activity.

There are also other causes of alopecia such as greasy or oily scalp due to seborrhea and the dandruff accompanying it, the present invention is not directed toward treating these causes of alopecia.

Hair growth enhancement and hair loss prevention treatments are known in the prior art. These include mixture of natural products, biological products, vasodilators and testosterone blockers. Some of these products are described in the following patents: In U.S. Pat. No. 5,183,817, Bazzano teaches how a combinations of retinoids and minoxidil-type compounds is more effective for hair growth. In U.S. Pat. No. 5,340,579, Casero discloses a composition comprising (a) mucopolysaccharides, (b) human umbilical cord extract, (c) tetrahydrofurfuryl nicotinate, and (d) excipients. In U.S. Pat. Nos. 5,512,275 and 5,609,858, Buck discloses a formulation for the treatment of androgenic alopecia, comprising liquor carbonis detergens in combination with spirits of camphor, castor oil, and isopropyl alcohol. In U.S. Pat. No. 5,972,345, Chizick et al., disclose a combination of saw palmetto extract, African pygeum extract, and stinging nettle extract. In U.S. Pat. No. 5,994,319, Hoke discloses using genetic material as an anti alopecia therapeutic. Hoke proposes using anti-sense oligonucleotides targeting 5-.alpha. reductases in conjunction with other hair growth enhancers. In U.S. Pat. No. 5,574,011, Tien discloses the use of a class of LHRH (Luteinizing hormone-releasing hormone, prostate cancer drug) analogs for treating male pattern baldness. Messenger in U.S. Pat. No. 6,020,327, discloses administering aromatase inhibitors to treat hair loss. Liao et al., disclose a class of anti-androgenic compounds in U.S. Pat. Nos. 5,422,371 and 5,605,929. U.S. Pat. No. 6,420,352 to Knowles, entitled “Hair loss prevention”, teaches Compositions to prevent or reduce hair loss, allowing the body to maintain normal, healthy hair growth, comprising a penetration enhancer together with a testosterone blocker or a vascular enhancer, or both. Nitrovasodilators such as Minoxidil has been shown to stimulate hair growth or inhibit the loss of hair in a number of patients beginning to develop androgenic alopecia. Archives in Dermatology, vol 125, August, 1989 discuss Endothelium-Derived Relaxing Factor (EDRF) and Minoxidil: Active Mechanisms in Hair Growth. It further describes a role for the nitroxide free radical (NO) in the control of vascular tone, platelet function, and in the central nervous system. The NO is apparently endothelium-derived relaxing factor, or EDRF, an endogenous compound probably accounting for the action of nitrovasodilators such as nitroglycerin. Because many other vasodilators act by increasing the endothelial production and release of EDRF, the elucidation of this system has caused a revolution in vascular physiology. Thus, minoxidil or (more likely) an active metabolite may be an EDRF agonist. Further, EDRF and minoxidil both activate guanylate cyclase,(1-3) an action thought to account for their common vasodilatory properties and one that is shared by many electronically activated compounds. Perhaps a separate action of EDRF on hair growth also explains minoxidil-induced hypertrichosis.

Minoxidil, a potent anti-hypertensive compound, has been found to promote hair growth when applied topically to the scalp, as discussed in U.S. Pat. Nos. 4,139,619 and 4,596,812 to Chidsey et al. Minoxidil is recognized as being somewhat effective in producing new vellus hair growth and sparse terminal hair growth. However, its effect is far from satisfactory in most subjects. Minoxidil is the generic name for 6-(1-piperidinyl)-2,4-pyrimidinediamaine 3-oxide or (2,4-diamino-6-piperidinopyrimidine- 3-oxide) and is currently available as Rogaine^{RTM}. Its preparation is disclosed in Anthony, W. C. et al., U.S. Pat. No. 3,382,247 (1968). Minoxidil is an anti-alopecia agent and its effectiveness in treating early male pattern baldness has been described in the prior art. See for example Olsen, E. A. et al., J. Am. Acad. Dermatol. 185 (1985); Novak, E., Int. J. Dermatol. 82 (1982). Minoxidil is believed to act by increasing vascular circulation to the hair follicle. It is certainly a radical nitric oxide (NO) donor which releases NO that activates the enzyme guanylate cyclase (sGC) which then causes the synthesis of the smooth muscle relaxant guanosine 3′,5′-monophosphate (cGMP), thereby promoting systemic vascular relaxation and dilation in order to increase vascular circulation and blood flow to hair follicles and hair bulbs. However, as soon as cGMP is produced another enzyme called phosphodiesterase 5 (PDE5) tends to degrade it and eliminate it. That is one of the reasons why topical Minoxidil is known to have certain shortcomings. It is effective in only about eight percent of adult male users. It produces “lanugo,” or baby-type, hair, which is relatively thin. Further, and perhaps most significantly, after approximately 30 months of continuous use, minoxidil shows a sharp drop in effectiveness probably due to local abundance of PDE5 which tends to fight the synthesis of cGMP which is needed as a vasodilator to enhance blood flow and vascular circulation to hair follicles and hair bulbs.

Thus, the present invention proposes the addition of cGMP specific PDE5 inhibitors such as sildenafil citrate (Viagra^{RTM}) to remedy the problem.Testosterone Inhibitors on the other hand are inhibitors of steroid metabolism, particularly those that inhibit the conversion of testosterone to dihydro testosterone, have shown effects on hair cycles, including inhibition of hair loss. One class of enzymes targeted by these inhibitors is the steroid 5-.alpha. reductases. The majority of body and facial hair growth is stimulated by androgens. However, the growth of scalp hair is genetically programmed to be inhibited by 5.alpha.-dihydrotestosterone (“DHT”) in individuals who exhibit a hereditary pre-disposition to baldness. Ebling, Dermatol. Clin. S. 467 (1987); Lucky, 4 Biochem. Soc. Transc. 597 (1988); Brodland et al., 47 Cutis 173 (1991). Reducing testosterone with a 5.alpha.-reductase enzyme produces DHT. These inhibitors apparently work by inhibiting the reduction of testosterone to DHT, as DHT is considered to be the more active form. The use of a combination of finasteride and minoxidil has demonstrated that, in combination, these two drugs increased the rate of hair growth when compared to either compound administered alone. Minoxidil used in conjunction with effectors of steroid metabolism, leads to enhanced hair growth and decreased rates of hair loss. It is of note that androgenic alopecia or common baldness represents more than 99 percent of all cases of hair loss. The prior art does show that the combination of Minoxidil with other chemicals can enhance hair growth but does not describe nor suggest the combination of the present invention for topically enhancing hair growth or diminishing hair loss or alopecia. Therefore, the aim of the present invention is to describe a new topical drug for enhancing hair growth or diminishing hair loss or alopecia, which comprises a mixture of a nitric oxide (NO) donor and a cyclic guanosine 3′,5′-monophosphate (cGMP) specific phosphodiesterase type 5 (PDE5) inhibitor such as sildenafil citrate (Viagra^{RTM}) in a dermatologically acceptable solution mix for topical treatment of hair loss and hair growth.

In he next section a summary of the said invention is presented.

### SUMMARY OF INVENTION

The present invention describes a finding that combination of nitric oxide (NO) releasing agents such as nitroglycerin, L-arginine, isosorbide dinitrate, sodium nitroprusside (sodium nitroferricyanide), or pyrimidine (also known as Minoxidil or Rogaine^{RTM}), or alternatively 2,4-Diamino-6-piperidinopyrimidine 3-N-oxide and a cyclic guanosine 3′,5′-monophosphate (cGMP) specific phosphodiesterase type 5 (PDE5) inhibitor such as sildenafil citrate (Viagra^{RTM}) or 1-[[3-(6,7-dihydro-1-methyl-7-oxo-3-propyl-1H-pyrazolo[4,3-d]pyrimidin-5-yl)-4-ethoxyphenyl]sulfonyl]-4 methylpiperazine citrate, in an dermatologically acceptable solution mix, when administered topically in proper concentration, works synergistically to promote systemic vascular relaxation, enhanced vascular circulation and blood flow to the hair bulbs and follicles. Thus, cGMP-PDE-5 inhibitors such as sildenafil citrate or mixtures thereof in combination with vasodilators such as minoxidil and/or minoxidil-type compounds are synergistically effective in stimulating or increasing the rate at which hair grows on mammalian skin. Thus, one can convert vellus hair growth to terminal hair growth, and treat alopecias due to organic dysfunction of the hair follicle and bulb by topical application to the hair and hair follicles and bulbs and to the skin adjacent thereto cGMP-PDE-5 inhibitors such as sildenafil citrate or mixtures thereof in combination with vasodilators such as minoxidil and/or minoxidil-type compounds. Preparations such as lotions, creams, shampoos, and the like, containing the aforementioned compounds as the active ingredients, can be applied topically to the skin, hair and/or follicles for this purpose. Oral administration of the mixture may also be used.

The basic mechanism at work here is that mammalian hair follicles can respond to nitrovasodilators in a much more pronounced manner if the systemic vascular relaxation and dilation of the scalp tissues and veins due to the presence of incipient cyclic guanosine 3′,5′-monophosphate (cGMP) is not impeded by the presence of cGMP specific degrading enzyme of phosphodiesterase type 5 (PDE5). Therefore, as a first step in solving this problem a chemical inhibitor of phosphodiesterase type 5 (PDES) must be present. In addition to such presence, however, the synthesis of smooth tissue relaxant cyclic guanosine 3′,5′-monophosphate (cGMP) must be preceded by the presence of activated enzyme guanylate cyclase (sGC) which then causes the synthesis of guanosine monophosphate (cGMP) to take place. However, the enzyme guanylate cyclase (sGC) needs to be activated by the presence of an active nitric oxide radical (NO) or chemicals that are capable of releasing NO in such tissues. Thus, the presence of a NO releasing agents such as nitrovasodilators will be necessary. The mixture can synthesize NO in areas that cannot themselves produce it, thereby promoting systemic vascular relaxation and dilation in order to enhance blood flow and vascular circulation to hair follicles and hair bulbs.

### DETAILED DESCRIPTION

According to the invention it has been found that cyclic guanosine 3′,5′-monophosphate (cGMP) specific phosphodiesterase type 5 (PDE5) inhibitors such as sildenafil citrate (Viagra^{RTM}) or mixtures thereof in combination with vasodilators such as minoxidil and/or minoxidil-type compounds (Rogaine^{RTM}) are synergistically effective in stimulating or increasing the rate at which hair grows on mammalian skin, converting vellus hair to terminal hair growth, and treating alopecias due to organic dysfunction of the hair follicle by topical application to the hair and hair follicles and to the skin adjacent thereto. Preparations such as lotions, creams, shampoos, and the like, containing the aforementioned compounds as the active ingredients, can be applied topically to the skin, hair and/or follicles for this purpose. Oral administration of the mixture may also be used. The present invention proposes a topical drug for enhancing hair growth or diminishing hair loss or alopecias, which comprises a mixture of a nitric oxide (NO) donor such as nitrovasodilators like minoxidil, nitroglycerin, L-arginine, isosorbide dinitrate, or nitroprusside, and a cyclic guanosine 3′,5′-monophosphate (cGMP) specific phosphodiesterase type 5 (PDE5) inhibitor such as sildenafil citrate (Viagra^{RTM}) in an ophthalmologically acceptable solution mix. In this manner there will be increased blood circulation to the hair follicles.

Nitric oxide is a gaseous molecule produced in the body through the enzymatic degradation of L-arginine. “Nitric oxide donor compound” means any compound (including small molecules, polymers, etc.) that releases nitric oxide or which acts as a substrate leading to the formation of nitric oxide. A wide variety of nitric oxide donor compounds are available for the release/production of nitric oxide, including the following: Organic nitrates such as nitroglycerine. O-nitrosylated compounds also known as O-nitroso compounds or in some cases organic nitrites). S-nitrosylated compounds also known as S-nitroso compounds or S-nitrosothiols compounds such as glutathione, S-nitrosylated derivatives of captopril, S-nitrosylated-proteins/peptides, S-nitrosylated oligosaccharides and polysaccharides. Nonoates compounds such as piperazines 2 and diazeniumdiolates. Inorganic nitroso compounds such as sodium nitroprusside. Sydnonimines. L-arginine (which does not release NO directly, but rather is an enzyme substrate which leads to the formation of nitric oxide in vivo). 1,3-(nitrooxymethyl)phenyl 2-hydroxybenzoateisosorbide dinitrateand pyrimidine (also known as Minoxidil or Rogaine ^{RTM}) A substance released by the endothelium, “endothelium derived relaxing factor” (EDRF), is now known to be nitric oxide (NO) or a compound, which liberates NO. This substance relaxes vascular smooth muscle, inhibits platelet aggregation, inhibits mitogenesis and proliferation of cultured vascular smooth muscle, and leukocyte adherence. NO may have other effects, either direct or indirect, on the various cells associated with vascular walls and degenerative diseases of the vessel. Thus, the presence of NO releasing agents such as nitrovasodilators will be necessary for enhancing blood flow and vascular circulation to the hair follicles and hair bulbs. The mixture can synthesize NO in areas that cannot themselves produce it, thereby promoting systemic vascular relaxation and dilation in order to enhance hair growth. The release of NO stimulates the activation of an enzyme necessary for the synthesis of guanosine 3′ 5′-cyclic monophosphate, (cGMP) in a target cell by directly activating the soluble isoform of enzyme guanylate cyclase (sGC). NO then activates the enzyme guanylate cyclase, which results in increased levels of synthesis of cyclic guanosine monophosphate (cGMP), which should escape degradation by phosphodiesterase, type 5 (PDE5) enzyme. Thus, the presence of a guanosine 3′,5′-monophosphate (cGMP) specific phosphodiesterase type 5 (PDE5) inhibitor such as sildenafil citrate (Viagra^{RTM}) which is designated chemically as 1-[[3-(6,7-dihydro-1-methyl-7-oxo-3-propyl-1-H-pyrazolo[4,3-d]pyrimidin-5-yl)-4-ethoxyphenyl]sulfonyl]-4 methylpiperazine citrate, is necessary. Some suitable cGMP PDE5 inhibitors for the use according to the present invention include: 1-[[3-(6,7dihydro-1-methyl-7-oxo-3-propyl-1H-pyrazolo[4,3-d]pyrimidin-5-yl)-4 ethoxyphenyl]sulphonyl]-4-methylpiperazine (sildenafil); 3-ethyl-5-[5-(4-ethylpiperazin-1-ylsulphonyl)-2-(2-methoxyethoxy) pyridin-3-yl]-2(pyridin-2-yl)methyl-2,6-dihydro-7H-pyrazolo[4,3-d]pyrimidin-7-one; 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpip-erazine; 5-(5-Acetyl-2-propoxy-3-pyridinyl)-3-ethyl-2-(1-isopropyl-3-azetidinyl)-2,6-dihydro -7H-pyrazolo[4,3-d]pyrimidin-7-one 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one; The suitability of any chosen cGMP PDE5 inhibitor can be readily determined by evaluation of its potency and selectivity by evaluation of its toxicity, absorption, metabolism, pharmacokinetics, etc in accordance with standard pharmaceutical practice. The Preferred cGMP PDE5 inhibitor here is Sildenafil citrate of Pfizer also known as Viagra^{RTM}. The preferred NO donor chemical here is Minoxidil type such as (Rogaine^{RTM}).The basic biochemistry mechanism at work here is that mammalian hair follicles can respond to nitrovasodilators in a much more pronounced manner if the systemic vascular relaxation and dilation of the scalp tissues and veins due to the presence of incipient cyclic guanosine 3′,5′-monophosphate (cGMP) is not impeded by the presence of cGMP specific degrading enzyme of phosphodiesterase type 5 (PDE5).

Therefore, as a first step in solving this problem a chemical inhibitor of phosphodiesterase type 5 (PDE5) must be present. In addition to such presence, however, the synthesis of smooth tissue relaxant cyclic guanosine 3′,5′-monophosphate (cGMP) must be preceded by the presence of activated enzyme guanylate cyclase (sGC) which then causes the synthesis of guanosine monophosphate (cGMP) to take place. However, the enzyme guanylate cyclase (sGC) needs to be activated by the presence of an active nitric oxide radical (NO) or chemicals that are capable of releasing NO in such tissues. That is why the presence of a NO releasing agents such as nitrovasodilators is essential. The mixture can synthesize NO in areas that cannot themselves produce it, thereby promoting systemic vascular relaxation and dilation in order to enhance blood flow and vascular circulation to hair follicles and hair bulbs. The release of NO stimulates the synthesis of guanosine 3′ 5′-cyclic monophosphate, (cGMP) in a target cell by directly activating the soluble isoform of enzyme guanylate cyclase (sGC). NO then activates the enzyme guanylate cyclase, which results in increased levels of synthesis of cyclic guanosine monophosphate (cGMP) which escapes degradation by phosphodiesterase type 5 (PDE5) enzyme, in the presence of a guanosine monophosphate (cGMP) specific phosphodiesterase type 5 (PDE5) inhibitor such as sildenafil citrate (Viagra^{RTM}) which is designated chemically as 1-[[3-(6,7-dihydro-1-methyl-7-oxo-3-propyl-1H-pyrazolo[4,3-d]pyrimidin-5-yl)-4-ethoxyphenyl]sulfonyl]-4 methylpiperazine citrate. Historically, nitrovasodilators such as minoxidil have been found to increase blood flow and vascular circulation to hair bulbs and follicles and enhance hair growth, as discussed before. However, the combined effect of nitrovasodilators to promote the release of nitric oxide (NO) to activate the enzyme guanylate cyclase (sGC) for the increased level of synthesis of guanosine 3′ 5′-cyclic monophosphate, (cGMP) in the presence of cGMP specific PDE5 inhibitors in a concurrent fashion to enhance vascular circulation and blood flow to hair bulbs and follicles and enhance hair growth and diminish hair loss is a novel concept that has not been yet discussed in the pertinent literature. In addition to nitric oxide (NO) releasing agents such as nitroglycerin or C3H5N3O9, sodium nitroprusside (sodium nitroferricyanide) or Na2 Fe(CN)5NO-2H2O, pyrimidine (also known as Minoxidil or Rogaine^{RTM}), or C9H15N5O or alternatively 2,4-Diamino-6-piperidinopyrimidine 3-N-oxide, (Ignarro et al., J. Pharmacol. Exp. Ther., 218, 739-749 (1981); Ignarro,Annu. Rev. Pharmacol. Toxicol., 30, 535-560 (1990); Kruszyna et al., Toxicol. Apol. Pharmacol., 91, 429-438 (1987); Wilcox et al., Chem. Res. Toxicol., 3, 71-76 (1990)), are other NO releasing compounds of interest to the present invention. The reader is referred to U.S. Pat. No. 6,379,660 to Saavedra, et al. Entitled “Nitric oxide-releasing 1-[(2 carboxylato)pyrrolidin-1-yl]diazen-1-ium-1,2-diolates and composition comprising same” which discusses a polymeric composition capable of releasing nitric oxide under physiological conditions which includes a biopolymer, such as a peptide, polypeptide, protein, oligonucleotide or nucleic acid, to which is bound a nitric oxide-releasing functional group and pharmaceutical compositions comprising the polymeric composition, U.S. Pat. No. 6,391,895 to Towart, et al., entitled “Nitric oxide releasing chelating agents and their therapeutic use”, that discusses chelating agents, in particular dipyridoxyl and aminopolycarboxylic acid based chelating agents, and their metal chelates, when linked directly or indirectly to at least one nitric oxide releasing moiety, or when use in combination with nitric oxide or a nitric oxide releasing moiety. It must be noted that minoxidil without a cGMP-PDE5 inhibitor produces a different kind of hair than does minoxidil used with a cGMP-PDE5 inhibitor.

It is believed that topical minoxidil such as Rogaine^{RTM} (commercially available from Pharmacia & Upjohn Inc., Bridgewater, N.J.) without a cGMP-PDES inhibitor such as sildenafil citrate (Viagra^{RTM}, commercially available from Pfizer) results in thin, baby-like, temporary hair, called “lanugo” hair. The present invention will result in good, coarse, “terminal” hair, hair, which is normal, permanent adult hair. Accordingly, the present invention provides: A dermatological hair growth enhancing/hair loss diminishing composition containing a combination of NO releasing agents and cGMP-PDE5 inhibitors; The hair follicles vasodilatory composition containing NO releasing agents and a method for enhancing hair growth and diminishing hair loss, comprising administering a pharmaceutically effective amount of a compound containing NO releasing agents and cGMP-PDE5 inhibitors; The NO donor cGMP-PDES inhibitor compound of the present invention may be mixed with a dermatological carrier vehicle. A variety of safe carriers may be used for this invention. These carriers are simply cosmetically safe, medically safe solvents or emulsions for the active ingredients. The carrier liquid should not adversely and significantly chemically react with the active ingredients of the present invention. For example, various combinations of propylene glycol, water and isopropyl alcohol may be used as liquid carriers. The carrier can optionally provide other functions in addition to simply dissolving the active ingredients. For example, one can use a moisturizing carrier. For a moisturizing or moisture-retaining carrier, one can use a combination of water, mineral oil, petrolatum, lanolin, sorbitol solution, stearic acid, lanolin alcohol, cetyl alcohol, triethanolamine, dimethicone, propylene glycol, methylparaben, ethylparaben, propylparaben, fragrance, methyldibromo glutaronitrile and others. It will be desirable to use a dermatological vehicle containing one or more sunscreens. Sunscreens, and vehicles containing sunscreen compounds, are widely known in the art. See for example U.S. Pat. No. 4,522,807 to Kaplan, entitled “Substantive topical compositions” that teaches a highly substantive topical composition in the form of an oil-in-water emulsion containing an octadecene -1/maleic anhydride copolymer. The foregoing invention has been described in some detail by way of illustration and example for purposes of clarity of understanding to make it readily apparent to those of ordinary skill in the related art that certain changes and modifications may be made thereto without departing from the spirit or scope of the appended claims.

In the next section the claims are described.

1- method for enhancing hair growth or diminishing hair loss or alopecia, in mammals, comprising administering topically to the skin a mixture of a nitric oxide (no) donor such as nitrovasodilators like minoxidil-like compounds such as (Rogaine^{RTM}), nitroglycerin, L-arginine, isosorbide dinitrate, or nitroprusside, and a cyclic guanosine 3′,5′-monophosphate (cGMP) specific phosphodiesterase type 5 (PDE5) inhibitor such as sildenafil citrate (Viagra^{RTM}) in a dermatologically acceptable solution mix.

2- Method according to claim 1, wherein said topical dermatological compound is in the form of an aqueous solution or suspension, or in the form a gel, a shampoo, an ointment or a cream in a pharmaceutically acceptable dermatological vehicle or carrier to be applied to the mammalian skin.

3- Method according to claim 1, wherein the No releasing agent in said dermatological mix is a organic nitrate such as nitroglycerine.

4- Method according to claim 1, wherein the No releasing agent in said dermatological mix is a O-nitrosylated compound also known as O-nitroso compounds or in some cases organic nitrites.

5- Method according to claim 1, wherein the No releasing agent in said dermatological mix is a S-nitrosylated compound also known as S-nitroso compounds or S-nitrosothiols compounds such as glutathione.

6- Method according to claim 1, wherein the No releasing agent in said dermatological mix is S-nitrosylated derivatives of captopril.

7- Method according to claim 1, wherein the No releasing agent in said dermatological mix is S-nitrosylated-proteins/peptides.

8- Method according to claim 1, wherein the No releasing agent in said dermatological mix is S-nitrosylated oligosaccharides and polysaccharides.

9- Method according to claim 1, wherein the No releasing agent in said dermatological mix is a Nonoate compounds such as piperazines 2 and diazeniumdiolates.

10- Method according to claim 1, wherein the No releasing agent in said dermatological mix is an inorganic nitroso compound such as sodium nitroprusside.

11- Method according to claim 1, wherein the No releasing agent in said dermatological mix is Sydnonimines.

12- Method according to claim 1, wherein the No releasing agent in said dermatological mix is L-arginine (which does not release NO directly, but rather is an enzyme substrate which leads to the formation of nitric oxide in vivo).

13- Method according to claim 1, wherein the No releasing agent in said dermatological mix is 1,3-(nitrooxymethyl)phenyl 2-hydroxybenzoate isosorbide dinitrate.

14- Method according to claim 1, wherein the No releasing agent in said dermatological mix is pyrimidine (also known as Minoxidil or Rogaine^{RTM}).

15- Method according to claim 1, wherein the cGMP specific PDE-5 inhibitor in said dermatological mix is (sildenafil) also known as 1-[[3-(6,7dihydro-1-methyl-7-oxo-3-propyl-1H-pyrazolo[4,3-d]pyrimidin-5-yl)-4 ethoxyphenyl]sulphonyl]-4-methylpiperazine.

16- Method according to claim 1, wherein the cGMP specific PDE-5 inhibitor in said dermatological mix is sildenafil citrate, (Viagra^{RTM}) also known as 1-[[3-(6,7dihydro-1-methyl-7-oxo-3-propyl-1H-pyrazolo[4,3-d]pyrimidin-5-yl)-4 ethoxyphenyl]sulphonyl]-4-methylpiperazine citrate.

17- Method according to claim 1, wherein the cGMP specific PDE-5 inhibitor in said dermatological mix is 3-ethyl-5-[5-(4-ethylpiperazin-1-ylsulphonyl)-2-n-propoxyphenyl]-2-(pyridin-2yl)methyl-2,6-dihydro-7H-pyrazolo[4,3-d]pyrimidin-7-one.

18- Method according to claim 1, wherein the cGMP specific PDE-5 inhibitor in said dermatological mix is 1-{6-ethoxy-5-[3-ethyl-6,7-dihydro-2-(2-methoxyethyl)-7-oxo-2H-pyrazolo[4,3-d]pyrimidin-5-yl]-3 pyridylsulphonyl}-4-ethylpip-erazine.

19- Method according to claim 1, wherein said topical dermatological mix is in the form of an aqueous solution and further contains one or more tonicity adjusting agents, one or more buffers and one or more antioxidants.

20- Method according to claim 1, wherein said topical dermatological mix further contains one or more antimicrobial agents.

21- The composition according to claim 1, wherein said dose is in pill form for oral administration.

22- The method according to claim 1, wherein said topical dermatological mix further contains one or more combinations of NO donors and cGMP PDE5 inhibitors.

23- The method according to claim 1, wherein said topical dermatological mix further contains one or more weight or volume percentage combinations of NO donors and cGMP PDE5 inhibitors.

24- A composition according to claim 1 wherein said composition also includes a pharmaceutically effective vehicle for said compound.

25- A composition according to claim 1 used in veterinary preparations or feeds to increase the rate of growth of fur (pelt) in certain fur bearing animals and to retard shedding and molting.
